(19) United States
(12) Patent Application Publication
(10) Pub. No.: US 2006/0188587 A1
(43) Pub. Date: Aug. 24, 2006

(54) Cranberry processes and products

(76) Inventors: Martin F. Berry, Raynham, MA (US); Katherine G. Haight, Mattapoisett, MA (US); Donald C. Weber, Marion, MA (US); Harold L. Mantius, North Kingstown, RI (US); Luther H. Leake, Mattapoisett, MA (US); Rod Serres, Duxbury, MA (US)

Correspondence Address: FISH & RICHARDSON PC, P.O. BOX 1022, MINNEAPOLIS, MN, 55440-1022, US

(21) Appl. No.: 11/406,599

(22) Filed: Apr. 19, 2006

Related U.S. Application Data
(63) Continuation of application No. 09/447,023, filed on Nov. 22, 1999, now abandoned.
Application No. 09/447,023 is a continuation-in-part of application No. 09/188,436, filed on Nov. 9, 1998, now abandoned.
Application No. 09/447,023 is a continuation of application No. PCT/US99/26373, filed on Nov. 9, 1999.

(51) Int. Cl. A61K 36/45; A23L 2/00

(52) U.S. Cl. 424/732; 426/599

(57) ABSTRACT

In embodiments, cranberries are utilized at certain phases of development, prior to full red color development, which are typically characterized by an acid profile conducive to producing flavorful, high-value products, such as juices. At select phases of development, the berries have a quinic acid content which is similar to fully red berries but a citric acid content which is much higher than fully red berries. As a result, the juice from these berries can be used with less need to add citric acid or citric acid juice, like lemon juice, to modify flavor. The berries at the select phase of development typically have a light color, from pink to white, which can be quantified based on the anthocyanin level in the fruit or the resulting juice.

### CROSS REFERENCE TO RELATED APPLICATIONS

This application is a continuation-in-part of U.S. Ser. No. 09/188,436 filed Nov. 9, 1998 and a continuation of PCT/US99/______ filed Nov. 9, 1999, entitled “Cranberry Processes and Products”, by Martin F. Berry, Katherine G. Haight, David C. Weber, Harold L. Mantius and Luther H. Leake, the entire contents of both are incorporated herein by reference.

### FIELD OF THE INVENTION

This invention relates to cranberry processing and products.

### BACKGROUND

Cranberries and the products made with them, such as juice, are typically characterized by a relatively strong astringent aftertaste, which is unpalatable to many consumers. An acid which is characteristic of cranberry and contributes to the aftertaste is quinic acid. To reduce the aftertaste, the juice is sometimes diluted and blended with other juices, sweeteners (e.g., sucrose), or additives (e.g., citric acid), to create a more palatable product. Cranberries and cranberry products are also typically characterized as red in color.

The red color of cranberries develops as cranberries mature during cultivation. Very young cranberries have a green color. In the next phase of development, the green turns to a white or pale yellow color. Finally, the white or pale yellow changes to a deep red color at full maturity in most berries. In a minority of cranberries, red color may never develop due to lack of direct sunlight or other factors.

The most commercially desirable berries have a uniform, deep red color. As a result, growers tend to leave berries in their bogs as long as possible to a time just prior to the onset of the historically predicted fruit-damaging frost so that most of the berries reach the deep red color stage. Harvest takes place in a short period, usually eight weeks or less. The short harvest time requires extensive investment in harvesting equipment. In the event that frost occurs early, growers wet the berries using sprinkler systems to reduce the risk of losing much of their crop.

Berries are harvested to be processed for sale either as fresh produce or to be processed into juice, concentrate, or other food products such as cranberry sauce. The berries harvested for the fresh produce market are usually shipped to a processor. Typically, the berries are graded and separated by color, either by visual inspection or optical scanning equipment. The berries with the deepest red color are targeted for sale as fresh produce. The other fractions are either discarded or reblended and then made into products, such as juice, concentrate, or sauce. Berries harvested for further processing into juice, concentrate, or other food products are typically analyzed by lot for anthocyanin (red pigment) content, and the berry lots are blended so that juice, concentrate, or other food product meets minimum red color standards.

### SUMMARY OF THE INVENTION

This invention relates to utilizing cranberries at certain phases of development, preferably color development, which are conducive to producing flavorful, low-color, high-value products, such as juices and blended juice drinks. Embodiments may include one or more of the following advantages. At select phases of development, typically color development, berries may have a lower quinic acid to citric acid ratio and a citric acid content which is higher than fully ripe berries. As a result, the juice from these berries can be used to prepare pleasant-tasting, low color-cranberry products, with reduced aftertaste, and with less need to add citric acid or citric acid-containing juices, such as lemon juice, to modify flavor. The berries at the select phase of development typically have a light color, from pink to white, which can be quantified based on the anthocyanin level in the fruit or the resulting juice.

Since the light color cranberries have a high-value use, harvesting may be timed to optimize the yield of light color cranberries rather than full red cranberries in at least some bogs. This allows the grower to extend the length of the harvest, reducing capital equipment requirements and the risk of crop loss due to frost.

In a first aspect, the invention features a method of utilizing cranberries. The method includes cultivating cranberries to a stage of development in which about 40% or more of the cranberries have an acid profile as described herein, such as a juice citric acid level as described herein, preferably about 1.4% or greater, or about 1.7% or greater, or about 1.4-1.9%, or about 1.6-1.8%, and/or a juice anthocyanin level as described herein, preferably about 10 mg/100 ml or less, or about 1-10 mg/100ml or about 6-8mg/100 ml. The method includes harvesting the cranberries and processing the cranberries.

In another aspect, the invention features a method of utilizing cranberries, including cultivating cranberries in a bog, administering a color retarding agent, sorting the cranberries by color, collecting cranberries having an anthocyanin level or acid profile as described herein and processing the cranberries. In embodiments, the method includes administering the agent in a single application during the cultivating. The agent may be a surfactant.

In another aspect, the invention features a method of processing cranberries. The method includes inspecting cranberries to select cranberries having an acid profile and/or an anthocyanin content, as discussed, and processing the cranberries.

In other aspects, the invention features a cranberry juice having the color and/or acid profiles described herein. In embodiments, the juice features a citric acid level of about 1.4% or more, a quinic acid to citric acid ratio of less than about 0.85, and a juice anthocyanin level of about 10 mg/100 ml or less. The juice features a citric acid level of about 1.7% or more, a quinic acid to citric acid ratio of about 0.5-0.75, and a juice anthocyanin level of about 1-10 mg/100ml. The juice features a citric acid level of about 1.6-1.8%, a quinic acid to citric acid ratio of about 0.65-0.7, and a juice anthocyanin level of about 2-8 mg/100 ml. The juice features a citric acid level of about 1.4-2%, a quinic acid to citric acid ratio of 0.65-0.85 and a juice anthocyanin level of about 1.5-8mg/100 ml. In other aspects, the juice is a blended juice, including a cranberry juice as described herein, where the citric acid component of the blended juice is provided substantially solely by the cranberry.

Embodiments may include one or more of the following. About 80% or more of the berries have the citric acid level. The cranberries have a juice quinic acid to citric acid ratio of about 0.85 or less or a juice quinic to citric acid ratio of about 0.5-0.75. Processing includes inspecting the cranberries to separate the cranberries at the phase of development from cranberries not at the phase of development. Processing includes inspecting the cranberries based on color. Processing the cranberries includes producing a blended juice. The blended juice has a citric acid content contributed substantially solely by the cranberries. The blended juice has about 2-35% ofjuice from cranberries. The blended juice has an absorbance of 515 nm light of about 0.5 or less.

In another aspect, the invention features a blended juice or blended juice product including juice from Yellow Bell cranberries. In embodiments, the blended juice or blended juice product may have about 2-35% of juice from Yellow Bell cranberries. In embodiments, the blended juice or blended juice product has a juice anthocyanin level of about 10 mg/100 ml or less.

In another aspect, the invention features a cranberry food product comprising infused husks of Yellow Bell cranberries. In another aspect, the invention features a cranberry food product including a predetermined blend of cranberries including cranberries having an anthocyanin content of about 10 mg/100 ml or less, e.g. Yellow Bell cranberries. In embodiments, the food product is a blended juice or a blended juice product or an infused cranberry husk or a fresh fruit mixture or a dried cranberry.

In other aspects, the invention features a cranberry food product consisting essentially of Yellow Bell cranberries or including Yellow Bell cranberries. In another aspect the invention features a method of processing cranberries, including collecting Yellow Bell cranberries, and processing said cranberries to produce a food product. In embodiments, the food product is a blended juice, a blended juice product, a dried cranberry, an infused cranberry husk, a sauce, jam, or jelly.

In other aspects, the invention features blending cranberries with other cranberries of known characteristics, and/or with other ingredients, to produce food products with desirable color and/or chemical, e.g. acid characteristics.

Unless indicated otherwise acid levels and anthocyanin values are by weight of a solution standardized to a juice soluble solids of 7.5%. Soluble solids is a measure of solids content by weight, typically determined by refractive index. A suitable technique for soluble solids measurement is described in the protocol in Appendix 1, which follows the method in “Method 932.12: Solids (Soluble) in Fruits and Fruit Products, Refractometer Method”, Official Methods of Analysis of the Association of Official Analytical Chemists, vol. 2, Fifteenth Edition, Kenneth Helrich, ed., Association of Official Analytical Chemists, Inc., Arlington, Va., 1990, the entire contents of which is incorporated herein by reference. Data is standardized by measuring the soluble solids of expressed juice, dividing the measured soluble solids into 7.5 to obtain a standardization factor, and multiplying the analytical factors determined on the expressed juice by this standardization factor.

Further advantages, aspects and features follow.

### DESCRIPTION OF THE PREFERRED EMBODIMENTS

We first briefly describe the drawings.

Drawings

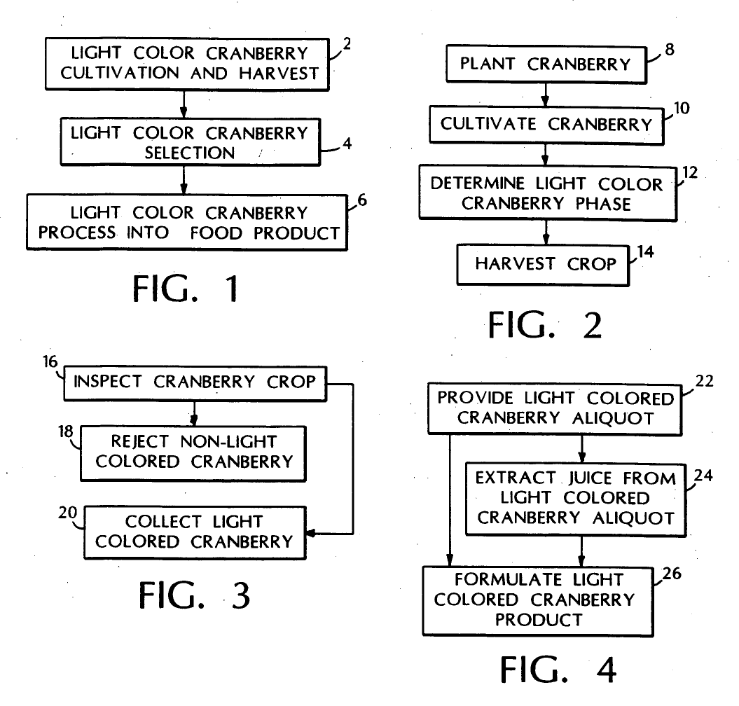

FIG. 1 is a flow diagram of growing, selecting and processing light color cranberries;

FIG. 2 is a flow diagram for growing and harvesting light colored cranberries;

FIG. 3 is a flow diagram for selecting light colored cranberries; and

FIG. 4 is a flow diagram for processing light colored cranberries.

### DESCRIPTION

Referring to FIG. 1, a flow diagram provides an overview of the utilization of light color cranberries. In the first step 2, light color cranberries may be cultivated and harvested. In the next step 4, the light color cranberries are selected. Particularly, the light color cranberries having the desirable characteristics, as discussed herein, are selected while other cranberries that may have been harvested, such as red berries, are separated. In the next step 6, the selected light color cranberries are processed into a food product, such as a juice.

Referring to FIG. 2, the steps of cultivating and harvesting light color cranberries are outlined. In the first step 8, the cranberries are planted. The cranberries that can be utilized include the American cranberry (Vaccinium macrocarpon) and related species including the European cranberry (Vaccinium oxycoccus), and the upland cranberry or lingonberry (Vaccinium vitis-idaea). A particular variety of American cranberry is the Stevens variety. Varieties may be selected that yield a particular natural acid profile or low color. For example, varieties with particularly high citric acid, a low quinic/citric ratio, and/or low color may be selected. For example, a low color variety is the Yellow Bell, grown in Maine. A variety may be genetically modified to enhance yield of light color berries or berries with the desirable acid characteristics, regardless of color. Other varieties are described in F. L. Caruso “Cranberry Cultivors” in Cranberry Productions: A Guide for Massachusetts, University of Massachusetts Extension Publication SP-127, p. 40 (1998), the entire contents of which is incorporated herein by reference.

In the next step 10, the cranberries are cultivated. The cranberries may be cultivated using commonly applied techniques. Alternatively, cultivation may be modified to produce a higher yield of berries that have desirable acid characteristic and/or low color. Modifications may include fertilization changes, temperature, and/or water availability. Light color berry yield may be enhanced by shading a bog, for example, with an awning. Administration of a color retarding agent may be used to enhance the yield of light color berries. Surface treatments may enhance light color berry yield. Surfactant treatments, using a surfactant in the organosilicone family, such as Break-thru (polyether-polymethylsiloxane copolymer, manufactured by Goldschmidt Chemical Corporation, Hopewell, Va.) can increase light color yield. The class of fungicides known as EBDC's (ethylene-bis-dithiocarbamates), including mancozeb, maned, zineb types, for example, Manzate (manufactured by E.I. DuPont de Nemours & Co., Wilmington, Del.), inhibit color development in cranberries when applied during the red color development phase. The influence of fungicides and certain environmental factors on color is discussed in De Moranville et al., “Influence of Weather on Cranberry Crop Production and Quality”, in Cranberry Production: A Guide for Massachusetts, H. A. Sandler, ed., University of Massachusetts Extension Publication SP-127, East Wareham, Mass., p. 14, 1997, the entire contents of which is incorporated herein by reference.

In the next step 12, the light color cranberry phase is determined. The desired cranberry phase can be determined by, for example, testing a sample of the berries periodically during the growing season for citric acid, quinic acid, and/or color. Determining the cranberry color is a preferred technique for determining when the cranberries are at the light color phase. The color inspection may be carried out visually by use of color comparison charts or by an experienced inspector. Alternatively, the inspection maybe carried out using optical comparitors. An example of a suitable optical comparitor is the Accuscan Optical Inspection System by Key Technology, Inc., Walla Walla, Wash. The color may be tested by producing a juice from sample berries (e.g., by pressing) and determining the anthocyanin content of the juice. Measurement of anthocyanin content in juice may be carried out according to Appendix 2. For a typical cranberry variety, the cranberry has reached a suitable phase when it has a juice anthocyanin level of around 10 mg/100 ml or less, preferably 8 mg/100 ml or less, more preferably about 3.5 mg/100 ml or less or in the range of 1-10 mg/100 ml or 6-8 mg/100 ml. Anthocyanin level of the whole berry may also be tested. The anthocyanin level in the whole berry is typically about 20 mg/100 g or less, (Typically, anthocyanin level in the berry measured in mg/100 g is about twice the value of the juice at 7.5% soluble solids measured in mg/100 ml). Berry anthocyanin level may be carried out as described in Appendix 3.

Alternatively, the desired light color cranberry phase may be determined based on the citric and quinic acid levels. The cranberries are typically suitable when the juice citric acid level is about 1.4% or more, preferably 1.5% or more, more preferably about 1.7% or more or in the range of 1.4-1.9% or 1.6-1.8%. The quinic acid to citric acid ratio may also be used. Typically the ratio is 0.85% or less, for example, around 0.75%. Combinations of anthocyanin levels, citric acid level, and quinic to citric ratio can also be used to determine the desirable development phase. While it is particularly desirable that the selected cranberries have a light color, cranberries with the acid profile desired herein can be used regardless of the color. The cranberries can be monitored during the growing season, e.g., monthly, during the earliest stages of growth, and then more frequently, e.g., weekly or daily, as time of harvest approaches.

The light color phase of the crop may also be determined based only on experience in the region in which the cranberries are grown. For example, in Wisconsin the Stevens variety fairly consistently reaches the light color phase about two to three weeks prior to the beginning of normal harvest time of the second or third week of October.

In the next step 14, the light color cranberry crop is harvested. Harvesting preferably takes place when about 40% or 50%, preferably about 80% or 90% or more of the cranberries in a given bog are in the light color cranberry phase.

Referring to FIG. 3, the light color cranberry selection procedure is discussed. In the first step 16, the berries in the harvested crop are inspected. The inspection criteria includes the same factors used to determine the development phase discussed above. The color of the cranberry is most preferably used as an indicator of which cranberries within the crop are suitable for further processing. Typically, each cranberry is inspected separately and either accepted as a light color cranberry or rejected as a non-light color cranberry. Alternatively, cranberries, or their juice, may be inspected in batches. Green cranberries may be included as part of the light-color cranberry fraction.

As indicated in FIG. 3, in step 20, cranberries which meet the selection criteria are collected for further processing where, in step 18, cranberries which do not meet the selection criteria are rejected. As a result, the cranberries which are selected have a substantially uniform color and/or acid profile characteristics. For example, the selected berries may have a quinic acid level comparable to red cranberries but a citric acid level that is higher than red cranberries. In addition, the selected berries also typically have a color which is much lighter than the non-selected berries. The berries that were rejected may be used in other cranberry products or discarded.

Referring to FIG. 4, the selected cranberries may be processed as follows. In a first step 22, an aliquot of the selected berries is provided. In the next step 24, juice may be extracted from the selected cranberry aliquot. Extraction of the juice can be achieved by a number of techniques. Examples include low temperature countercurrent techniques such as those described in Mantius et. al, U.S. Pat. No. 5,320,861, the entire contents of which is incorporated herein by reference. The Mantius '861 patent also describes steps for producing processed juices, which may also be applied to berries as described herein. The countercurrent extraction process may also produce a decharacterized cranberry husk or shell from which the juice was extracted. The husk or shell may also be used for infused fruit products. The expressed juice may also be extracted using a press. The juice may absorb comparatively weakly in the red region of the spectrum. For example, the juice may have an absorption of about 0.5 or less at 515 nm. Absorption can be measured in a 10% by volume dilution of the juice (0.75% solids by weight) in a pH 2.8 buffer (84.15% by volume of a 0.1 M citric acid solution with 15.85% of a 0.2 M sodium phosphate dibasic). Optical absorbance may be measured using a spectrophotometer, for example, the Hach DR/3000 from Hach Company, Loveland, Ohio.

In step 26, the selected cranberries are formulated into a food product, such as a pure cranberry juice, a pure blended juice, including cranberry and other juices, or a blended juice product with less than 100% juice by, for example, dilution with water, addition of sweetener, addition of acid, or addition of other juices. As FIG. 4 indicates, the light color cranberries can also be processed into products other than juice, such as concentrate, sauce, or infused food products which may be pure fruit products or products with non-fruit components. An advantage of the selected cranberry fraction is that it typically has a relatively high overall acid level and citric acid level, while having a quinic acid level more comparable to red cranberries. The extracted juice or food product from the selected cranberries can be flavored with other fruits or ingredients to produce a fruit juice or food product. The light color and pleasant flavor of the light color cranberries or the extracted juice from the selected cranberries facilitates the formulation of cranberry products with mild flavor and colors other than red.

Further embodiments are illustrated by the following examples.

### EXAMPLE 1

Samples of about 1 pound of previously harvested and frozen cranberries were sorted by hand into three visual color categories: red, pink, and white. The samples were pressed by a hydraulic piston press (Carver Instruments, Carver, Inc., Wabash, Inc.) to produce a pure juice. The juices from the sorted samples were analyzed for titratable acidity by titration to pH 8.1 with 0.01 M NaOH, as discussed in Appendix IV which follows the technique in “Method 942.15: Acidity (Titratable) of Fruit Products”, Official Methods of Analysis of the Association of Official Analytical Chemists, vol. two, Fifteenth Edition, Kenneth Helrich, ed., Association of Official Analytical Chemists, Inc., Arlington, Va., p. 918, 1990, the entire contents of which is incorporated herein by reference. The total acidity is expressed using the citric acid factor. Quinic, citric, and malic acids were determined by high pressure liquid chromatography (Coppola et al., “Authenticity of Cranberry Products Including Non-Domestic Varieties”, in Methods to Detect Adulteration of Fruit Juice Beverages, vol. 1, S. Nagy and R. L. Wade, eds., Agscience, Inc,. Auburndale, Fla., pp. 287-308, 1995). Juice anthocyanin level was determined by the method of Appendix 1. Finally, the dextrose/fructose ratio was determined by high pressure liquid chromatography (Coppola et al, supra.). The results are indicated in Table 1. The table also includes data for unsorted berries (see Coppola et al. supra) and from lemon juice (Dillon, A., “Fruit Juice Profiles”, in Methods to Detect Adulteration of Fruit Juice Beverages, vol. 1, S. Nagy and R. L. Wade, eds., Agscience, Inc., Auburndale, Fla., pp. 392-397, 1995).

TABLE 1

| | Unsorted | Visual | | Visual | |
|---|---|---|---|---|---|
| | Conventional | Sort | Visual Sort | Sort | Lemon |
| Parameter | Harvest* | Red* | Pink* | White* | Juice |
| Numbers of Samples | 385 | 16 | 5 | 32 | — |
| Anthocyanin (juice) | — | 19.01 | 7.4 | 2.01 | — |
| mg/100 ml | | | | | |
| % Titratable Acidity | 2.06(0.22) | 2.22(0.26) | 2.79(0.28) | 2.85(0.27) | 4.5(min.) |
| Soluble solids | 7.5 | 7.5 | 7.5 | 7.5 | 7.0(approx.) |
| (standardized) | | | | | |
| Soluble solids/Acid Ratio | 3.64 | 3.38 | 2.69 | 2.63 | 1.55(max) |
| % Quinic (juice) | 1.07(0.12) | 1.06(0.14) | 1.19(0.26) | 1.16(0.23) | — |
| % Malic (juice) | 0.78(0.08) | 0.90(0.07) | 0.89(0.09) | 0.77(0.08) | 0.15-0.41 |
| % Citric (juice) | 1.08(0.16) | 1.10(0.15) | 1.62(0.25) | 1.79(0.27) | 4.0-6.5 |
| Quinic/Citric (juice) | 1.00(0.12) | 0.99(0.20) | 0.75(0.20) | 0.68(0.23) | — |
| Dextrose/Fructose | — | 3.77 | 4.81 | 5.11 | — |

*Standard deviation given in parentheses

As the data indicate, light color berries, having an anthocyanin content of around 7.4, exhibited a citric acid level of around 1.62%, much more than red cranberries (1.10%). However, the light color cranberries had a quinic acid content of 1.19%, which is more comparable to that of red cranberries (1.06%). The enhancement of the citric acid is increased with cranberries that have an even lower anthocyanin content. For cranberries with an anthocyanin content of 2.01, the citric acid level was 1.79%. The quinic acid level was 1.16%.

### EXAMPLE 2

Juice products were formulated using red cranberries and light color cranberries. The characteristics of the expressed juice from the berries are provided in Table 2.

TABLE 2
juice characteristics

| | % Titratable | | | | |
|---|---|---|---|---|---|
| | Acidity | % Quinic | % Malic | % Citric | Color* |
| Cranberry | (juice) | (juice) | (juice) | (juice) | (juice) |
| Light Color | 3.19 | 0.89 | 0.65 | 1.65 | 0.317 |

| | | | |
|---|---|---|---|
| Red | 1.95 | Not Measured | 1.294 |
| (conventional | | | |
| harvest) | | | |

*Optical absorbance at 515 nm of juice diluted to 0.75% soluble solids with pH 2.8 buffer.

The juice products were formulated to identical titratable acid and solids levels. These levels were around 0.49% titratable acidity (expressed as % citric acid), and 12.2% solids (soluble solids). Formula A was formulated with the light color cranberry juice which was pressed from light color cranberries, and Formulas B, C, and D were formulated with conventionally harvested and pressed red cranberry juice. The juice content was determined based on a standardized solids content of 7.5% soluble solids. The beverage formulas are given in Table 3.

TABLE 3
Beverage Formulas

| | Formula (% w/w) | | | |
|---|---|---|---|---|
| Ingredient | Formula A | Formula B | Formula C | Formula D |
| Light Color Cranberry | 16.0 | — | — | — |
| Juice | | | | |
| Red Cranberry Juice | — | 16.0 | 16.0 | 25.3 |
| Citric Acid | — | 0.15 | — | — |
| Lemon Juice | — | — | 3.3 | — |
| Sugar | 12.0 | 12.0 | 12.0 | 11.6 |
| Water | to 100 | to 100 | to 100 | to 100 |

Formula A used 16% light color cranberry juice as the sole source of acid to achieve an titratable acidity content of about 0.49%. Formula B and C used 16% red cranberry juice and added citric acid and lemon juice, respectively, to meet the titratable acidity target and formula D used 9.3% additional red cranberry juice to meet the titratable acidity target. The characteristics of the beverages are described in Table 4.

TABLE 4
Characteristics of Beverages

| Characteristic | Formula A | Formula B | Formula C | Formula D |
|---|---|---|---|---|
| Titratable | 0.488 | .0486 | 0.486 | 0.478 |
| Acidity % | | | | |
| Soluble | 12.2 | 12.2 | 12.2 | 12.2 |
| Solids % | | | | |
| Color (visual) | light pink | red | cloudy red | dark red |
| Flavor | Citrus Tartness | Moderate | Lemon | Intense |
| Description | Very Mild | Cranberry | Moderate | Cranberry |
| | Cranberry | | Cranberry | |

As the results indicate, formula A using the light color cranberry juice, meets the titratable acid target without resorting to citrus additives or higher juice content. In addition, for formula A the sensory characteristics were indicated as clean, citrus tartness with a very mild cranberry flavor, and a very pale pink color.

### EXAMPLE 3

Twenty-five one meter square test sites were established on a bog of the Early Black variety in Rochester, Mass. Treatments with Break-thru [symbol] (a polyether-polymethylsiloxane copolymer exempt from food tolerance requirement, under 40 CFR 180.1001(c)) (Goldschmidt Chemical Corporation, 914 E. Randolph Road, Hopewell, Va. 23860) (0.1% v/v in tap water) and control (water only) were made in the equivalent of 144 gallons of water per acre with a stainless steel backpack CO₂ pressurized backpack sprayer to plots in a completely randomized design, with five replicates per treatment. All plots were surrounded by a 20 centimeter buffer strip. Three treatments received sprays on July 31, and two of these treatments received an additional, identical treatment on either 11 or 15 August.

The single application of surfactant significantly reduced Early Black color, as measured by total anthocyanin (TAcy) content when berries were harvested (table). Two surfactant treatments further inhibits color. The size of berries was reduced by two surfactant treatments, but not by a single treatment.

A random example of harvested berries from each plot was subjected to electronic color-sorting into five categories based on color: dark red, dark pink, medium pink, light pink and white. Consistent with the overall anthocyanin results, red berries were less common, and white berries were more common, with surfactant treatment.

TABLE 5

| Number of | | Berry categories by color (percent) | | | | |
|---|---|---|---|---|---|---|
| surfactant | Total | Dark | Dark | Medium | Light | |
| applications | Anthocyanin* | Red | Pink | Pink | Pink | White |
| Zero | 41.9 | 40.2 | 57.0 | 0.1 | 1.1 | 1.6 |
| One | 31.2 | 19.6 | 65.8 | 1.9 | 4.7 | 7.9 |
| Two | 25.0 | 18.4 | 51.4 | 2.7 | 5.7 | 21.7 |

*T values in mg/100 ml differ among all three treatments, General Linear Models Procedure, Ryan-Einot-Gabriel-Welsch Multiple Range Test, overall p < 0.0001. Unequal sample numbers: 15 for zero-, 5 for one-, and 10 for two-surfactant application. See SAS Institute Inc., SAS/STAT (v) User's Guide, Version 6, Fourth Edition, Vol. 2, Cavy NC: SAS Institute, Inc. 1989, the entire contents of which is incorporated herein by reference.

### EXAMPLE 4

The following involves the use of Yellow Bell cranberries, a low color variety found in Vinalhaven, Me. The Yellow Bell is a North American cranberry of the species Vaccinium macrocarpon Ait. It produces vines and uprights similar to typical cranberry varieties. The leaves are small and ellipsoid and the 3-year stems are covered with dehiscent bark. The fruit is bell-shaped and small, at approximately 0.5-1 g/berry. The fruit is yellow-colored upon maturity often with a pinkish blush developing near the pedicel over time after maturity. The chemical profile is provided in Table 6.

TABLE 6

| | | | Sorted/early |
|---|---|---|---|
| Character | Yellow Bell¹ | Typical red² | harvest white³ |
| Fruit | | | |
| TAcy of fruit (mg/100 g | 3 | 38 | 8 |
| fruit) | | | |
| Brix of fruit | 8.0° | 7.5° | 7.0° |
| Juice | | | |
| Brix (standardized) | 7.5° | 7.5° | 7.5° |
| Acidity (as citric w/v) | 2.4% | 2.1% | 2.8% |
| Quinic acid | 1.28% | 1.06% | 1.16% |
| Malic acid | 0.47% | 0.78% | 0.77% |
| Citric acid | 1.19% | 1.06% | 1.79% |
| Quinic/citric ratio | 1.08 | 1.00 | 0.68 |
| Quinic/malic ratio | 2.75 | 1.36 | 1.53 |
| Sucrose | 0.17% | 0.05% | <0.05% |
| Dextrose | 2.81% | 3.24% | 2.37% |
| Fructose | 0.66% | 0.90% | 0.48% |
| Dextrose/fructose ratio | 4.42 | 3.60 | 5.11 |
| Absorbance @ 515 nm | 0.05^{a} | 1.29^{b} | 0.32^{b} |
| Anthocyanin | 0.63^{a} | 16.17^{c} | 1.92^{d} |
| (mg/100 ml juice) | | | |

¹Average of fruit from two seasons, and one location.
²Average of data base across years, cultivars, and locations.
³Average of 32 samples from two years early harvest and sorting of fruit of various cultivars and locations.
^{a}Data from one season.
^{b}Data taken from Table 2 above.
^{c}Average of 21 samples from two seasons of fruit of various cultivars and locations. Standardized to 7.5° brix from a reading done on 2° juice.
^{d}Average of 32 samples from two seasons early harvest and sorting of fruit of various cultivars and locations. Standardized to 7.5° brix from a reading done on 2° juice.

The fruit is typically characterized as being yellow in color upon maturity. Total anthocyanin (TAcy) content of the fruit is about 3 mg/100 g compared to a TAcy value of 38mg/100 g for typical red-fruited varieties. Total anthocyanin of the juice is typically less than about 1 mg/100 ml. A value of 0.63mg/100 ml is indicated in the table. The Brix level of the mature yellow fruit is around 80 compared to mature red fruit at 7.5°. The acid profile is characteristic in being high in quinic acid (1.28%) and low in malic acid (0.47%). Thus, it has a high quinic to malic ratio (2.75:1) when compared to both red and white fruit from typically cultivated varieties. The citric acid content is also high (1.19%) but the quinic to citric ratio is comparable to red berries (approx. 1:1). The sugar profile is also characteristic with sucrose being higher (0.17%) and dextrose and fructose being lower than red fruit (2.81% and 0.66% respectively). The dextrose to fructose ratio is higher than red fruit (4.42:1).

The Yellow Bell can be processed into various types of food products which may be substantially pure Yellow Bell products or products that are a blend of Yellow Bell and other cranberries or ingredients. Examples of food products include juice, blended juice, blended juice products, sauces, jams, jellies, dried cranberries, and products based on the cranberry husk. For example, the low color from the Yellow Bell permits juice products where full red color is not desirable, including blended juices and blended juice products, including blends with low color cranberries described above and/or red cranberries. The Yellow Bell cranberries may be particularly advantageous for dried cranberry products where low color is desirable. The extracted husks of the fruit can also be used to make infused products as described in the Mantius '861 patent, supra. The yellow cranberries are also useful in sauces, e.g., mixed with low color and/or red cranberries to make a sauce of desired color. In addition, the yellow cranberries can be mixed with low color and/or red cranberries to form fresh fruit mixtures that are pleasing to the eye. Harvesting of Yellow Bell may involve assessing maturity on chemical analysis and not color development as is done for red cranberries. Separation of Yellow Bell cranberries, where co-cultivated with other berries, may be accomplished based on color and/or the berry shape. The Yellow Bell cranberries may also be monitored and selected at early stages of maturity to obtain desirable characteristics, e.g. acid profiles. Yellow Bell cranberries include the Yellow Bell type characterized above as well as progeny of Yellow Bell, e.g. crossbred or hybridized varieties such as high yielding varieties, that produce low color fruit or other characteristics such as chemical characteristics, of Yellow Bell. Seeds for Yellow Bell are available from the National Clonal Germplasm Repository in Corvallis, Oreg. (accession no. P1555028).

Still further embodiments are within the following claims:

What is claimed is:

1.-69. (canceled)

70. A cranberry food product comprising a predetermined blend of cranberries including cranberries having an anthocyanin content of about 10 mg/100 ml or less.

71. The food product of claim 70 comprising Yellow Bell cranberries.

72. The food product of claim 70 or 71 wherein the food product is a blended juice.

73. The food product of claim 70 or 71 wherein the food product is a blended juice product.

74. The food product of claim 70 or 71 wherein the food product is an infused cranberry husk.

75. The food product of claim 70 or 71 wherein the product is a fresh fruit mixture.

76. The food product of claim 70 or 71 wherein the food product is a dried cranberry.

77.-84. (canceled)
